(19) United States
(12) Patent Application Publication
(10) Pub. No.: US 2007/0008404 A1
(43) Pub. Date: Jan. 11, 2007

(54) Method and system for displaying stereoscopic image

(76) Inventor: Seijiro Tomita, Tokyo (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/555,655

(22) PCT Filed: May 7, 2003
(86) PCT No.: PCT/JP03/05708

§ 371 Date: Nov. 4, 2005

(51) Int. Cl. A61B 1/04; H04N 13/00

(52) U.S. Cl. 348/43; 600/111; 352/57

(57) ABSTRACT

When displaying a stereoscopic image, in order to display a natural stereoscopic image such that an area other than an area of attention is not displayed ahead of the focused area, an area of attention to be clearly displayed where an object to be focused exists is specified at a time of displaying two images to make a stereoscopic image, and gradation processing is performed on an area other than the area.

### BACKGROUND OF THE INVENTION

1. Field of the Invention

The present invention relates to a stereoscopic display method and device thereof which especially defines a specific area of attention and positively gradates an area deviated from the area.

2. Description of the Related Art

Conventionally, a plurality of photographing means has been applied to capture a real image which is acquired as stereoscopic image information by picking up an object (photographic subject), for example, with a camera and then adjusted to a visual property.

A binocular parallax is one of the means. The binocular parallax requires a setting of two cameras at a base length of naked eyes (for example, 72 mm). Besides, every value is set for picking up a picture by taking into consideration a range of visual angle of convergence of naked eye.

When displaying these images, a distance from an observer to an object that the observer recognizes and a proper parallax (lateral deviance) are supplied for display.

Consequently, it requires changing the display image according to a change in camera photographing position at photographing and observer's viewpoint.

When running a content of same photographing on a display of a different screen size, intersection of parallaxes on both sides and phenomenon that a background scene is seen ahead of a front area of attention have been happened.

That is to say that, as shown in FIG. 10, in the case where the photographic subjects A and B in the background C are photographed by two cameras 1 and 2, if observing acquired images 41 and 42, the image in the background C that is a background of the photographic subject A to be focused may be recognized ahead of the photographic subjects A and B.

In view of the current situation, the present invention was made. The purpose is to provide a stereoscopic display method and device thereof which can display a natural stereoscopic image that does not display an area other than a focused area ahead of the focused area.

### DISCLOSURE OF THE INVENTION

The present invention solves the above mentioned problems with the following means.

A first aspect of the present invention provides a stereoscopic image display method, wherein when displaying a stereoscopic image by displaying two images, an area of attention to be clearly displayed in that an object to be focused exists is specified and any other area is performed with gradation processing.

According to the aspect, an area other than the area of attention in which an object to be focused exists is gradated, and since an observer cannot acquire a clear image in the area, the area of attention is clearly displayed stereoscopically.

A second aspect of the present invention provides a stereoscopic image display method according to the first aspect, wherein a front area of a cross-point is defined as an area of attention and an area behind the cross-point is performed with gradation processing.

According to the aspect, the front area of the cross-point in which an object to be focused usually exists is defined as an area of attention, and an out-of-focus area where background and so forth without any other subject to be focused are displayed are performed with gradation processing. Since an observer cannot acquire a clear image about the area, the focused area is clearly displayed stereoscopically.

A third aspect of the present invention provides a stereoscopic image display method according to the first aspect, wherein an area of attention is defined as a peripheral domain of the in-focus area and any other area is performed with gradation processing.

According to the aspect, the peripheral domain of the in-focus in which an object to be focused usually exists is defined as an area of attention and the out-of-focus area in which background and so forth without any other subject to be focused are displayed is performed with gradation processing. Since an observer cannot acquire a clear image from this area, the focused area is clearly displayed stereoscopically.

A fourth aspect of the present invention provides a stereoscopic image display method according to the first aspect, wherein an object to be focused is extracted and a peripheral domain thereof is defined as an area of attention, and any other area is performed with gradation processing.

According to the aspect, a peripheral domain of an object to be focused is defined as an area of attention and an area where back ground and so forth without any other subject to be focused are displayed is performed with gradation processing. Since an observer cannot catch a clear image in the area, the focused area is clearly displayed stereoscopically.

A fifth aspect of the present invention provides a stereoscopic image display method according to the first aspect, in which an area of attention is specified by calculation of a distance to an object of each pixel that constitutes an image.

According to the aspect, calculation of the distance to the object of each pixel that constitutes an image enables to specify the object to be focused. In this manner, an area to be gradated can be defined.

A sixth aspect of the present invention provides a stereoscopic image display method according to any one of the aspects first to fifth, wherein gradation degree of gradation processing is increased with distance from an area of attention.

According to the aspect, since a change from the area of attention to the gradated area becomes natural, an observer can acquire a natural stereoscopic image.

A seventh aspect of the present invention provides a stereoscopic image display method according to any one of the aspects first to fifth, in which information of a photographed image is once stored in an image memory and then each treatment is performed based on the information of the stored image.

According to the aspect, since each treatment can be performed on elimination of once stored image afterwards, setting of an area of attention or gradation processing is not required in real time and treatment at a higher speed is not required.

An eighth aspect of the present invention provides a stereoscopic image display, wherein when displaying a stereoscopic image with using two images the stereoscopic image display is comprised of an area focus means which defines an area of attention to be clearly displayed where an object to be focused exists and a gradation processing means which carries out gradation on any other area.

According to the aspect, since the area of attention in which the object to be focused exists is defined by the area focus means and any other area is performed with the gradation processing means, an observer cannot acquire a clear image for the area and the focused area is clearly displayed stereoscopically.

An ninth aspect of the present invention provides a stereoscopic image display according to the eighth aspect, wherein an area focus means defines a front area of a cross-point as an area of attention and a gradation processing means gradates a backward area of the cross-point.

According to the aspect, the area focus means defines as an area of attention a front area of the cross-point where an object to be focused usually exists and the gradation processing means gradates an out-of-focus area where background and so forth without any other subject to be focused are displayed. Therefore, an observer cannot acquire a clear image for the area and the focused area is clearly displayed stereoscopically.

A tenth aspect of the present invention provides a stereoscopic image display according to the eighth aspect, wherein an area focus means defines a peripheral area of an in-focus area as an area of attention and a gradation processing means gradates any other area.

According to the aspect, the area focus means defines as an area of attention the peripheral area of the in-focus area where an object to be focused usually exists and the gradation processing means gradates the out-of-focus area where background and so forth without any other subject to be focused are displayed. Therefore, an observer cannot acquire a clear image for the area and the focused area is clearly displayed stereoscopically.

An eleventh aspect of the present invention provides a stereoscopic image display according to the eighth aspect, wherein an area focus means extracts an object to be focused and defines a peripheral area thereof as an area of attention, and a gradation processing means gradates any other area.

According to the area, the area focus means defines the peripheral area of the object to be focused as an area of attention and the gradation processing means gradates an area without any other subject to be focused where background and so forth are displayed. Therefore, an observer cannot acquire a clear image for the area and the focused area is clearly displayed stereoscopically.

A twelfth aspect of the present invention provides a stereoscopic image display according to the eighth aspect, in which an area focus means can specify an area of attention by calculating a distance to an object of each pixel that constitutes an image specifies an area of attention. According to the aspect, the area focus means can specify an object to be focused by calculating a distance to an object of each pixel at which is photographed. In this manner, a gradated area can be specified.

A thirteenth aspect of the present invention provides a stereoscopic image display according to any one of the aspects eighth to twelfth, wherein a gradation processing means increases gradation degree with distance from an area of attention. According to the aspect, the gradation processing means makes a change from the area of attention to a gradated area natural and an observer can acquire a natural stereoscopic image.

A fourteenth aspect of the present invention provides a stereoscopic image display according to any one of the aspects eighth to thirteenth, wherein information of a photographed image is once stored in an image memory and then each treatment is performed based on the information of the stored image.

According to the aspect, since each treatment by the area focus means and the gradation processing means can be performed on elimination of once stored information in the memory afterwards, setting of a area of attention or gradation processing is not required in real time and treatment at a higher speed is not required.

### BRIEF DESCRIPTION OF THE DRAWINGS

FIG. 1 is a block diagram showing a structure of a conversion device of stereoscopic image signal in accordance with the present invention.

FIG. 2 shows a flow chart showing an actuation of the conversion device of stereoscopic image signal shown in FIG. 1.

FIG. 3 is a diagram showing an area of attention and a gradated area of image.

FIG. 4 is an explanatory diagram showing a gradation processing of image.

FIG. 5 is a block diagram showing an example of a conversion device of stereoscopic image signal in accordance with the present invention.

FIG. 6 is a diagram explaining a condition of photographed object.

FIG. 7 is a diagram showing an example of an area of attention and a gradated area.

FIG. 8 is a diagram showing another example of an area of attention and a gradated area.

FIG. 9 is a diagram showing other example of an area of attention and a gradated area.

FIG. 10 is a diagram showing a stereoscopic image photographing apparatus to which the present invention is applied.

### DESCRIPTION OF THE PREFERRED EMBODIMENTS

Now, embodiments of a conversion method of stereoscopic image signal and device thereof of the present invention will be described by referring to the accompanying drawings.

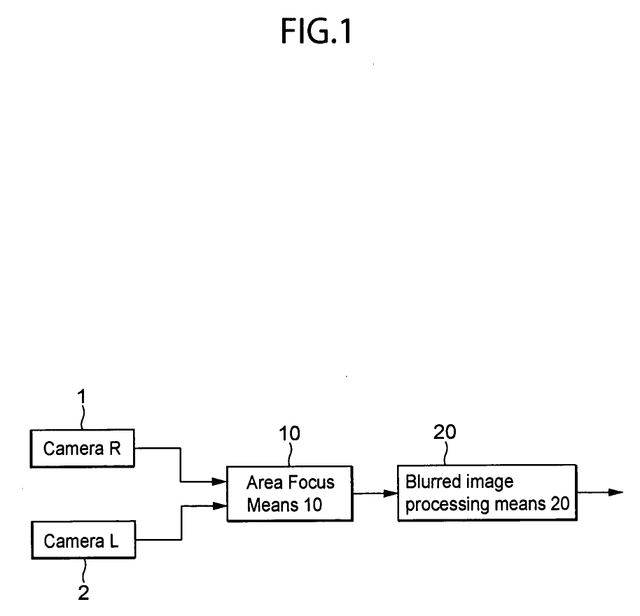

FIGS. 1 to 11 show an example of a constitution of a conversion method of stereoscopic image signal and device thereof in accordance with the present invention.

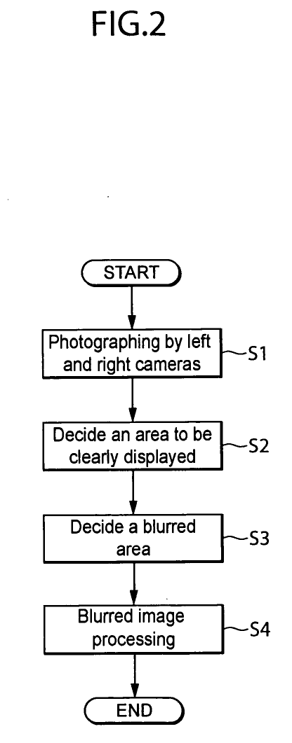

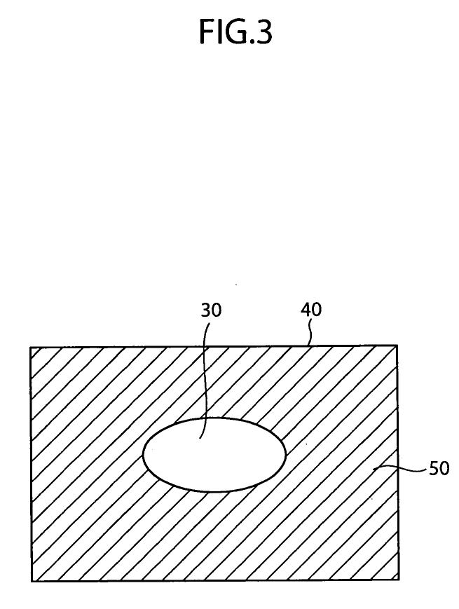

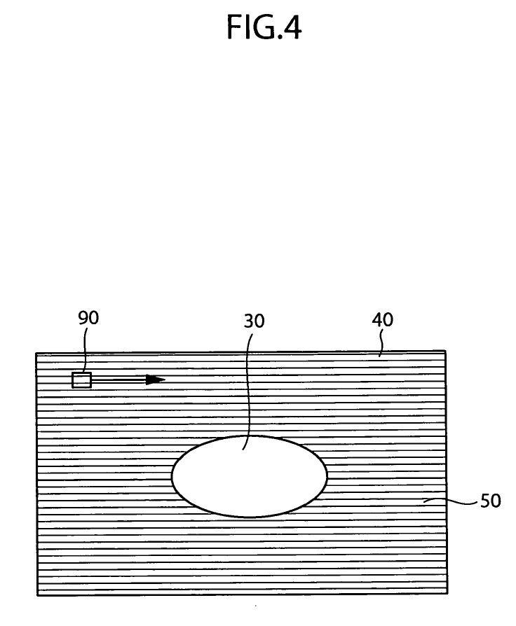

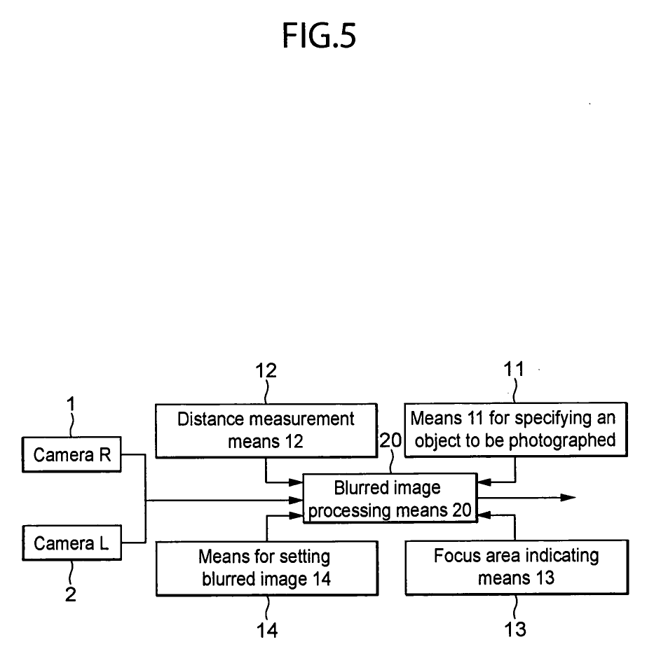

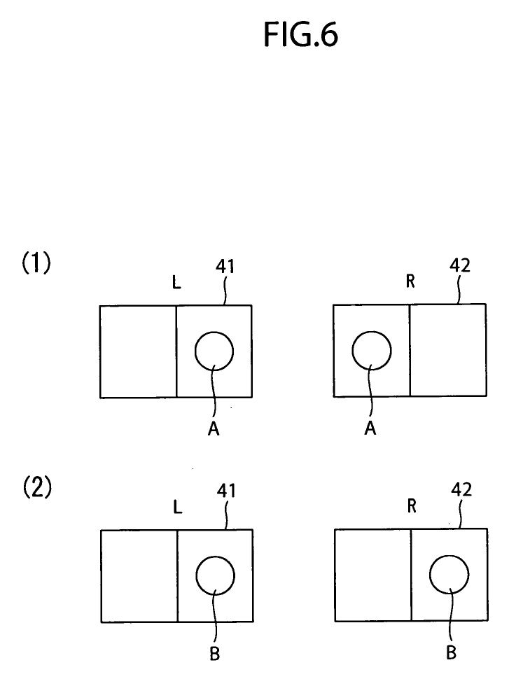

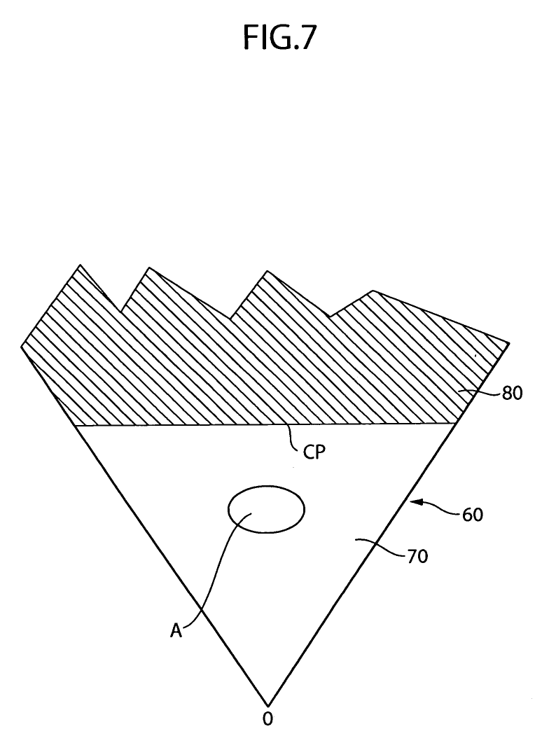

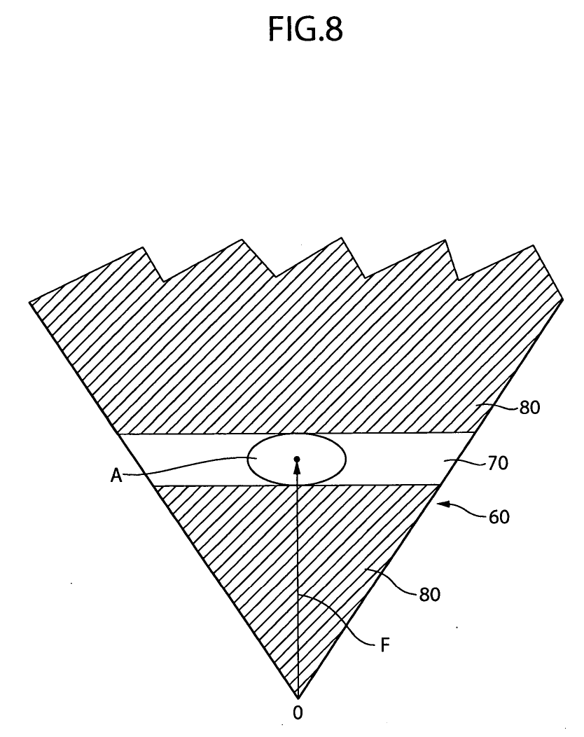

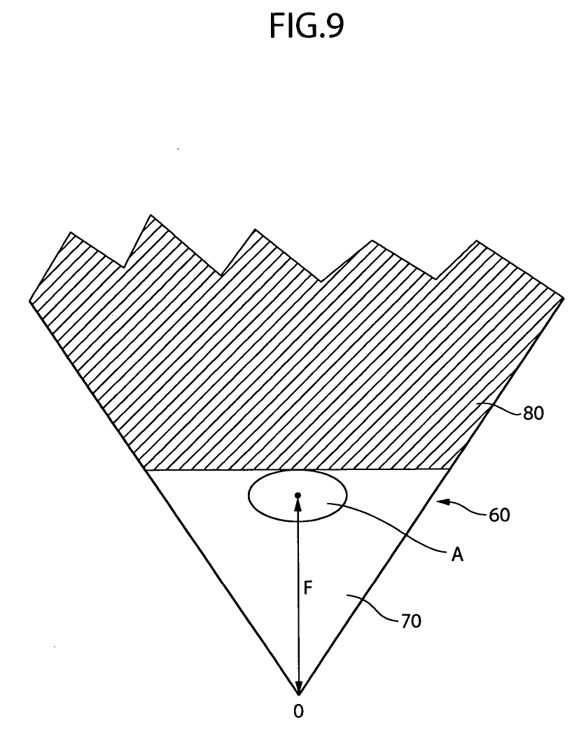

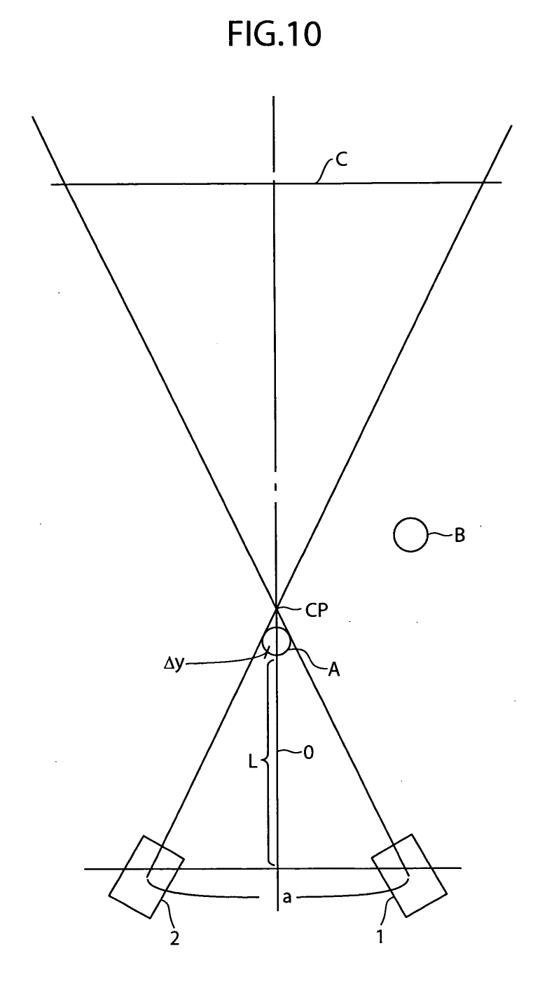

FIG. 1 is a block diagram showing a structure of a conversion device of stereoscopic image signal in accordance with the present invention; FIG. 2 shows a flow chart showing an actuation of the conversion device of stereoscopic image signal shown in FIG. 1; FIG. 3 is a diagram showing an area of attention and a gradated area of image; FIG. 4 is an explanatory diagram showing a gradation processing of image; FIG. 5 is a block diagram showing an example of a conversion device of stereoscopic image signal in accordance with the present invention; FIG. 6 is a diagram explaining a condition of photographed object; FIG. 7 is a diagram showing an example of an area of attention and a gradated area; FIG. 8 is diagram showing another example of an area of attention and a gradated area; FIG. 9 is a diagram showing other example of an area of attention and a gradated area; and FIG. 10 is a diagram showing a stereoscopic image photographing apparatus to which the present invention is applied.

In the present embodiment, a conversion device of stereoscopic image signal is basically comprised of an area focus means 10 which is connected to a right side camera 1 and a left side camera 2 and a gradation processing means 20.

In the present embodiment, the area focus means 10 defines an area of attention to be clearly displayed where an object to be focused (photographic subject) exists, when displaying a stereoscopic image by using two images photographed with the above two cameras 1 and 2.

The above gradation processing means 20 conducts a gradation processing on an area other than the above area of attention.

Process flow chart with the conversion device of stereoscopic image signal in accordance with the embodiment is shown in FIGS. 2, 3 and 4. Namely, two cameras of a right side camera 1 and a left side camera 2 perform photographing (S1). Then, the area focus means 10 defines an area of attention 30 to be clearly displayed in each image 40 acquired by the photographing (S2). In this manner, an area other than the area of attention to be gradated (gradated area 50) is defined (S3). Then, the gradation processing means 20 performs gradation on the gradated area.

As shown in FIG. 4, the gradation processing of each pixel of the gradated area 50 is performed using a known gradation filter 90, for example, such as Sobel filter, Laplacian filter and Gaussian filter. In this case, if gradation degree is increased with distance from the area of attention, a change from the area of attention 30 to the gradated area 50 becomes natural and an observer can acquire a natural stereoscopic image. These gradation degrees can be performed by changing filter size coefficient on software side and so forth.

Next, the following is an explanation of the area of attention by the area focus means 10 in accordance with the embodiment.

In this embodiment, as shown in FIG. 10, two cameras 1 and 2 are located with a distance d so that each optical axis crosses at the cross-point (CP).

Further, as shown in FIG. 5, the area focus means 10 is comprised of a subject to be photographed specifying means 11 which specifies a subject to be photographed, a distance measurement means 12 which measures a distance to the object to be focused, an area of attention indicating means 13 which indicates a size and so forth of the area of attention and a gradated condition setting means 14 which sets a gradation type, a gradation degree and so forth.

In a conversion device of stereoscopic image signal, specification of the area of attention can be decided with various methods.

A first method is a method that decides an area of attention based on cross-point (CP) information. As shown in FIG. 7, this defines the front side of the cross-point (CP) in the vision 60 as the area of attention 70 and the backward area from the cross-point (CP) as the gradated area 80. Namely, it can be said to be a method for defining an area of attention by whether the phase of the acquired image is located on the same phase or opposite phase. As shown in FIGS. 6 and 10, the method is in a same manner that this regards a portion of same phase as an area of attention and an opposite phase is defined as a gradated area. Here, a same phase means that subjects are asymmetrically located on the both sides of the central line which runs through a cross-point of the image (See FIG. 6(1)), and an opposite phase means that subjects are symmetrically located on the both sides of the central line which runs through a cross-point of the image (See FIG. 6(2)).

A second method is a method which regards a distance F to a focus object A, namely, a position 70 that cameras 1 and 2 focus on as an area 70 of attention and a front area and a backward area of the area of attention as gradated areas 80, 80. In this case, a distance L to the focus object A and a phase lag Δy from the axis O can be calculated in the following method.

Namely, in the case of the left camera below formulas are established.
y_{LP}/{(Δy/cos θ_{L})+[(Δz−Δy tan θ_{L})sin θ_{L}]}=f/{(zc/cos θ_{L})−[(Δz−Δy tan θ_{L})sin θ_{L}]} (Formula 1)
Here, f means a focus distance of lens for photographing means.

In the case of the right camera, similar formulas are established.

And,

if the cameras are fixed at θ_{L}=θ_{R}=θ, formula 1 is described in a simple way as below.
y_{LP}/{(Δy/cos θ)+[(Δz−Δy tan θ)sin θ]}=f/{(zc/cos θ)−[(Δz−Δy tan θ)sin θ]} (Formula 2)

From FIG. 4, since a triangle formed by f, yₗₚ and O_{y} and a triangle formed by f, P and Q are of plesiomorphism,

y_{LP}/‘A’=f/‘B’ is established.

Here, if ‘c’, ‘d’, ‘e’ and ‘f’ are calculated in a condition of ‘A’=‘c’+‘d’ and ‘B’=‘e’−‘f’, the below formula is drawn.
‘c’=(Δy/cos θ)
‘d’=(Δz−Δy tan θ)/sin θ
‘e’=(zc/cos θ)
‘f’=(Δz−Δy tan θ)·cos θ

If ‘c’ to ‘f’ are assigned to y_{L}/‘A’=f/‘B’,

the below formula is given.
y_{LP}/{(Δy/cos θ)+[(Δz−Δy tan θ)sin θ]}=f/{(zc/cos θ)−[(Δz−Δy tan θ)cos θ]} (Formula 3)

From these two formulas (Formulas 2 and 3), Δz and Δy may be drawn.

Here, since tan A is a field angle as well as a constant, it can be calculated or figured out by a regular measurement in advance. Further, the below figure of 756 is number of elements from the center of the CCD photographing element to the both right and left distal ends. This figure can be properly changed by alternating the number of photographing elements and the starting distal point for calculation (for example, putting a starting distal point on the left distal end).

Further, ‘R’ is calculated by the formula:
[(Δz+Δy tan θ)sin θ−(Δy/cos θ)]/[{z/cos θ−[(Δz+Δy tan θ)/cos θ]} tan A]=+x_{R}/756; and

‘L’ is calculated by the formula:
[(Δz−Δy tan θ)sin θ+(Δy/cos θ)]/[{z/cos θ−[(Δz−Δy tan θ)/cos θ]} tan A]=+x_{L}/756.

‘R’ becomes:
756(Δz+Δy tan θ)sin θ−[(756·Δy)/cos θ]=[(+z·x_{R}·tan A)/cos θ]−{[x_{R}(Δz+Δy tan θ)tan A]/cos θ}
756·tan θ·sin θ−(756/cos θ)+[(x_{R}·tan θ·tan A)/cos θ]Δy
=+756·Δz·sin θ+[(z·x_{R}·tan A)/cos θ]−[(x_{R}·Δz·tan A)/cos θ]

‘L’ becomes:
756(Δz−Δy tan θ)sin θ+[(756·Δy)/cos θ]=[(+z·x_{L}·tan A)/cos θ]−{[(x_{L}(Δz+Δy tan θ)tan A)/cos θ]
−756·tan θ·sin θ+(756/cos θ)−{[−(x_{L}·tan θ·tan A)]/cos θ}}Δy
=−756·Δz·sin θ+[(z·x_{L}·tan A)/cos θ]−[(x_{L}·Δz·tan A)/cos θ]
[−756·Δz·sin θ+{[tan A·x_{R}(z−Δz)]/cos θ}]/{756·tan θ·sin θ+[(−756+x_{R}·tan θ·tan A)/cos θ]}=[−756·Δz·sin θ+{[tan A·x_{L}(+z−Δz)]/cos θ}]/{−756·tan θ·sin θ+[(756−x_{L}·tan θ·tan A)/cos θ]}
‘K’=−756·Δz·sin θ
‘M’=(−756+x_{R}·tan θ·tan A)/cos θ
‘N’=(756−x_{L}·tan θ·tan A)/cos θ
‘O’=756·tan θ·sin θ+[(−756+x_{R}·tan θ·tan A)/cos θ]
‘P’=−756·tan θ·sin θ+[(756−x_{L}·tan θ·tan A)/cos θ]
{[−‘Q’·Δz+‘R’x_{R}(z−Δz)]/‘O’}={[−‘Q’·Δz+‘R’x_{L}(z−Δz)]/‘P ’}
−‘Q’‘P’Δz+‘R’‘P’x_{R}·z−‘R’‘P’x_{R}·Δz=−‘Q’‘O’·Δz+‘O’‘R’x_{L}·z−‘O’‘R’x_{L}·Δz
(−‘Q’‘P’−‘R’‘P’·x_{R}+‘O’‘R’x_{L}+‘Q’‘O’)Δz=+‘O’‘R’x_{L}·z−‘R’‘P’x_{R}·z=(‘O’‘R’x_{L}−‘R’‘P’x_{R})z
‘S’=−‘Q’‘P’−‘R’‘P’·x_{R}+‘O’‘R’x_{L}+‘Q’‘O’
‘O’=‘O’‘R’x_{L}−‘R’‘P’x_{R}

Here,

x_{R} and x_{L} show deviance of image, and

Z shows cross-point, and

answer to be calculated is Δz

‘R’ is calculated by the below formula.
756(Δz+Δy tan θ)sin θ−[(756·Δy)/cos θ]=[(z·x_{R}·tan A)/cos θ]−{[x_{R}(Δz+Δy tan θ)tan A]/cos θ}
756·Δz+[(x_{R}·tan A)/cos]·Δz=[(z·x_{R}·tan A)/cos θ]−[(x_{R}·Δy tan θ·tan A)/cos θ]−756·Δy·tan θ·sin θ+[(756·Δy)/cos θ]
{(756·cos θ+x_{R}·tan A)/cos θ}Δz=(z·x_{R}·tan A−x_{R}·Δy·tan θ·tan A−756·Δy·sin² θ+756·Δy)/cos θ

‘L’ is calculated by the below formula.
756(Δz−Δy tan θ)sin θ+[(756·Δy)/cos θ]=[(z·x_{L}·tan A)/cos θ]−{[x_{L}(Δz−Δy tan θ)tan A]/cos θ}
756·Δz+[(x_{L}·tan A)/cos θ]·Δz=[(z·x_{L}·tan A)/cos θ]+[(x_{L}·Δy tan θ·tan A)/cos θ]−756·Δy·tan θ·sin θ−[(756·Δy)/cos θ]
{(756·cos θ+x_{L}·tan A)/cos θ}Δz=(z·x_{L}·tan A+x_{L}·Δy·tan θ·tan A−756·Δy·sin² θ+756·Δy)/cos θ

Δy is also calculated by the basic formula. In this embodiment, Δy shows a deviance from the center.
(z·x_{R}·tan A−x_{R}·Δy·tan θ·tan A−756·Δy·sin² θ+756·Δy)/[(756·cos θ)+(x_{R}·tan A)]=(z·x_{L}·tan A+x_{L}·Δy·tan θ·tan A+756·Δy·sin² θ−756·Δy)/[(756·cos θ)+(x_{L}·tan A)]
[z·x_{R}·tan A+(−x_{R}·tan θ·tan A−756 sin² θ+756)Δy]/‘L’=[z·x_{L}·tan A+(x_{L}·tan θ·tan A+756·sin² θ−756)Δy]/‘M’
‘L’=(z·x_{R}·tan A−x_{R}·Δy·tan θ·tan A−756·Δy·sin² θ+756·Δy)/[(756·cos θ)+(x_{R}·tan A)]
‘M’=(z·x_{L}·tan A+x_{L}·Δy·tan θ·tan A+756·Δy·sin² θ−756·Δy)/[(756·cos θ)+(x_{L}·tan A)]
‘N’=−x_{R}·tan θ·tan A−756 sin² θ+756
‘O’=x_{L}·tan θ·tan A+756·sin² θ−756
‘Q’=z·x_{R}·tan A
‘R’=z·x_{L}·tan A
‘M’‘Q’+‘M’‘N’Δy=‘L’‘R’+‘L’‘O’Δy
{‘M’‘N’−‘L’‘O’}Δy=(‘L’‘R’−‘M’‘Q’)
=‘S’/‘T’

From the above, distance L from left and right images of photographing elements to a center of subject and Δy of deviance are calculated.

In the present embodiment, if the result is previously stored in a table, values of L and Δy can be output immediately at a time of acquiring an image.

Further, correction volume of aberration on the optical element is previously stored in the table. The value can be appropriately changed in conformity with the correction volume of lens and so forth to be used.

Next, a third method is a method which regards as an area of attention 70 a distance F to a focus object A, namely, a front side of the position 70 in that cameras 1 and 2 focus on, and a backward area of the area of attention 70 as a gradated area 80.

Further, irrespective of the above method, an area of attention can be fixed. That is to say that the above methods can be combined.

Moreover, calculation of information of two images enables to calculate a distance to a stereoscopic image of each pixel, and then a precise area of attention can be defined.

Further, information of a photographed image can be once stored in an image memory and each treatment can be performed based on the information of the stored image. In this case, setting of an area of attention and gradation processing in real time are not required and a treatment at a higher speed is not required.

As above, according to a conversion device of stereoscopic image signal in accordance with the present embodiment, since an area other than the focused area is gradated at display, an observer can concentrate himself/herself on the image in the area to be focused and enjoy watching. Besides, burden on eyes or brain of the observer can be reduced and help reduce physical fatigue accompanied by viewing stereoscopic image.

These treatments are extremely useful for practical use of stereoscopic display. Application to a 3D broadcasting and a 3D processing soft are of benefit.

### INDUSTRIAL APPLICABILITY

A first aspect of the present invention is to provide a stereoscopic image display method, wherein when displaying a stereoscopic image by displaying two images, an area of attention to be clearly displayed in that an object to be focused exists is specified and any other area is performed with gradation processing.

According to the aspect, an area other than the area of attention in which an object to be focused exists is gradated, and since an observer cannot acquire a clear image in the area, the area of attention is clearly displayed stereoscopically.

A second aspect of the present invention is to provide a stereoscopic image display method according to the first aspect, wherein a front area of a cross-point is defined as an area of attention and an area behind the cross-point is performed with gradation processing.

According to the aspect, the front area of the cross-point in which an object to be focused usually exists is defined as an area of attention, and an out-of-focus area where background and so forth without any other subject to be focused are displayed is performed with gradation processing. Since an observer cannot acquire a clear image about the area, the focused area is clearly displayed stereoscopically.

A third aspect of the present invention is to provide a stereoscopic image display method according to the first aspect, wherein an area of attention is defined as a peripheral domain of the in-focus area and any other area is performed with gradation processing.

According to the aspect, the peripheral domain of the in-focus in which an object to be focused usually exists is defined as an area of attention and the out-of-focus area in which background and so forth without any other subject to be focused are displayed is performed with gradation processing. Since an observer cannot acquire a clear image from this area, the focused area is clearly displayed stereoscopically.

A fourth aspect of the present invention is to provide a stereoscopic image display method according to the first aspect, wherein an object to be focused is extracted and a peripheral domain thereof is defined as an area of attention, and any other area is performed with gradation processing.

According to the aspect, a peripheral domain of an object to be focused is defined as an area of attention and an area where back ground and so forth without any other subject to be focused are displayed is performed with gradation processing. Since an observer cannot catch a clear image in the area, the focused area is clearly displayed stereoscopically.

A fifth aspect of the present invention is to provide a stereoscopic image display method according to the first aspect, in which an area of attention is specified by calculation of a distance to an object of each pixel that constitutes an image.

According to the aspect, calculation of the distance to the object of each pixel that constitutes an image enables to specify the object to be focused. In this manner, an area to be gradated can be defined.

A sixth aspect of the present invention is to provide a stereoscopic image display method according to any one of the aspects first to fifth, wherein gradation degree of gradation processing is increased with distance from an area of attention.

According to the aspect, since a change from the area of attention to the gradated area becomes natural, an observer can acquire a natural stereoscopic image.

A seventh aspect of the present invention is to provide a stereoscopic image display method according to any one of the aspects first to fifth, in which information of a photographed image is once stored in an image memory and then each treatment is performed based on the information of the stored image.

According to the aspect, since each treatment can be performed on elimination of once stored image afterwards, setting of an area of attention or gradation processing is not required in real time and treatment at a higher speed is not required.

An eighth aspect of the present invention is to provide a stereoscopic image display, wherein when displaying a stereoscopic image with using two images the stereoscopic image display is comprised of an area focus means which defines an area of attention to be clearly displayed where an object to be focused exists and a gradation processing means which carries out gradation on any other area.

According to the aspect, since the area of attention in which the object to be focused exists is defined by the area focus means and any other area is performed with the gradation processing means, an observer cannot acquire a clear image for the area and the focused area is clearly displayed stereoscopically.

An ninth aspect of the present invention is to provide a stereoscopic image display according to the eighth aspect, wherein an area focus means defines a front area of a cross-point as an area of attention and a gradation processing means gradates a backward area of the cross-point.

According to the aspect, the area focus means defines as an area of attention a front area of the cross-point where an object to be focused usually exists and the gradation processing means gradates an out-of-focus area where background and so forth without any other subject to be focused are displayed. Therefore, an observer cannot acquire a clear image for the area and the focused area is clearly displayed stereoscopically.

A tenth aspect of the present invention is to provide a stereoscopic image display according to the eighth aspect, wherein an area focus means defines a peripheral area of an in-focus area as an area of attention and a gradation processing means gradates any other area.

According to the aspect, the area focus means defines as an area of attention the peripheral area of the in-focus area where an object to be focused usually exists and the gradation processing means gradates out-of-focus area where background and so forth without any other subject to be focused are displayed. Therefore, an observer cannot acquire a clear image for the area and the focused area is clearly displayed stereoscopically.

An eleventh aspect of the present invention is to provide a stereoscopic image display according to the eighth aspect, wherein an area focus means extracts an object to be focused and defines a peripheral area thereof as an area of attention, and a gradation processing means gradates any other area.

According to the area, the area focus means defines the peripheral area of the object to be focused as an area of attention and the gradation processing means gradates an area without any other subject to be focused where background and so forth are displayed. Therefore, an observer cannot acquire a clear image for the area and the focused area is clearly displayed stereoscopically.

A twelfth aspect of the present invention is to provide a stereoscopic image display according to the eighth aspect, in which an area focus means can specify an area of attention by calculating a distance to an object of each pixel that constitutes an image specifies an area of attention.

According to the aspect, the area focus means can specify an object to be focused by calculating a distance to an object of each pixel at which is photographed. In this manner, a gradated area can be specified.

A thirteenth aspect of the present invention is to provide a stereoscopic image display according to any one of the aspects eighth to twelfth, wherein a gradation processing means increases gradation degree with distance from an area of attention.

According to the aspect, the gradation processing means makes a change from the area of attention to a gradated area natural and an observer can acquire a natural stereoscopic image.

A fourteenth aspect of the present invention is to provide a stereoscopic image display according to any one of the aspects eighth to thirteenth, wherein information of a photographed image is once stored in an image memory and then each treatment is performed based on the information of the stored image.

According to the aspect, since each treatment by the area focus means and the gradation processing means can be performed on elimination of once stored information in the memory afterwards, setting of an area of attention or gradation processing is not required in real time and treatment at a higher speed is not required.

1. A stereoscopic image display method, wherein when displaying a stereoscopic image by displaying two images, an area of attention to be clearly displayed in that an object to be focused exists is specified to be a front area of a cross-point and a backward area of the cross-point is performed with gradation processing.

2. (canceled)

3. A stereoscopic image display method according to claim 1, wherein an area of attention is defined as a peripheral domain of the in-focus area and any other area is performed with gradation processing.

4. A stereoscopic image display method according to claim 1, wherein an object to be focused is extracted and a peripheral domain thereof is defined as an area of attention, and any other area is performed with gradation processing.

5. A stereoscopic image display method according to claim 1, in which an area of attention is specified by calculation of a distance to an object of each pixel that constitutes an image.

6. A stereoscopic image display method according to claim 1, wherein gradation degree of gradation processing is increased with distance from an area of attention.

7. A stereoscopic image display method according to claim 1, in which information of a photographed image is once stored in an image memory and then each treatment is performed based on the information of the stored image.

8. A stereoscopic image display, wherein when displaying a stereoscopic image with using two images the stereoscopic image display is comprised of an area focus means which defines that an area of attention to be clearly displayed where an object to be focused exists is a front area of a cross-point and a gradation processing means which carries out gradation on a backward area of the cross-point.

9. (canceled)

10. A stereoscopic image display according to claim 8, wherein an area focus means defines a peripheral area of an in-focus area as an area of attention and a gradation processing means gradates any other area.

11. A stereoscopic image display according to claim 8, wherein an area focus means extracts an object to be focused and defines a peripheral area thereof as an area of attention, and a gradation processing means gradates any other area.

12. A stereoscopic image display according to claim 8, in which an area focus means can specify an area of attention by calculating a distance to an object of each pixel that constitutes an image specifies an area of attention.

13. A stereoscopic image display according to claim 8, wherein a gradation processing means increases gradation degree with distance from an area of attention.

14. A stereoscopic image display according to claim 8, wherein information of a photographed image is once stored in an image memory and then each treatment is performed based on the information of the stored image.

15. A stereoscopic image display method according to claim 3, wherein gradation degree of gradation processing is increased with distance from an area of attention.

16. A stereoscopic image display method according to claim 4, wherein gradation degree of gradation processing is increased with distance from an area of attention.

17. A stereoscopic image display method according to claim 5, wherein gradation degree of gradation processing is increased with distance from an area of attention.

18. A stereoscopic image display method according to claim 3, in which information of a photographed image is once stored in an image memory and then each treatment is performed based on the information of the stored image.

19. A stereoscopic image display method according to claim 4, in which information of a photographed image is once stored in an image memory and then each treatment is performed based on the information of the stored image.

20. A stereoscopic image display method according to claim 5, in which information of a photographed image is once stored in an image memory and then each treatment is performed based on the information of the stored image.
